(19) United States
(12) Patent Application Publication
(10) Pub. No.: US 2019/0076461 A1
(43) Pub. Date: Mar. 14, 2019

(54) MATERIALS AND METHODS FOR TREATING HYPOXIC CONDITIONS

(71) Applicant: MitoGenetics, LLC, Farmingdale, NY (US)

(72) Inventors: Richard M. Kream, Huntington, NY (US); George B. Stefano, Melville, NY (US)

(21) Appl. No.: 16/084,775

(22) PCT Filed: Mar. 13, 2017
(86) PCT No.: PCT/US17/22017

§ 371 Date: Sep. 13, 2018

Related U.S. Application Data
(60) Provisional application No. 62/307,931, filed on Mar. 14, 2016.

(51) Int. Cl. A61K 31/715; A61K 31/095; A61K 33/00; A61K 36/81; A61K 9/20; A61P 9/10; A23L 33/105; A23L 33/16; A23L 33/21

(52) U.S. Cl. CPC A61K 31/715; A61K 31/095; A61K 33/00; A61K 36/81; A61K 9/20; A61P 9/10; A23V 2250/21; A23L 33/16; A23L 33/21; A23V 2002/00; A23V 2200/326; A23V 2250/51; A23L 33/105

(57) ABSTRACT

This document provides materials and methods for treating hypoxic conditions (e.g., tissue ischemia). Compositions containing a potato polysaccharide preparation and organic selenium can be used to increase or maintain mitochondrial nitric oxide formation. Compositions provided herein can also contain inorganic nitrate.

### CLAIM OF PRIORITY

This application claims priority to U.S. Provisional Application Ser. No. 62/307,931, filed on Mar. 14, 2016. The disclosure of the prior application is considered part of the disclosure of this application, and is incorporated in its entirety into this application.

### TECHNICAL FIELD

This document relates to methods and materials for treating hypoxic conditions (e.g., tissue ischemia). For example, this document relates to compositions containing a potato polysaccharide preparation and organic selenium to increase or maintain mitochondrial nitric oxide (NO) formation. In some cases, compositions provided herein also can contain inorganic nitrate.

### BACKGROUND

The etiology and persistence of major metabolic disorders afflicting millions of humans worldwide involve a functional pathophysiological coupling of systemic pro-inflammatory processes and tissue hypoxia, indicating that ischemic/hypoxic perturbations in oxygen delivery represent significant physiological challenges to overall cellular and multiple organ system viability. Mechanistically, reciprocal triggering of multiple ischemic/hypoxic and pro-inflammatory events, if not corrected, will promote pathophysiological amplification leading to a deleterious cascade of bio-senescent cellular and molecular signaling pathways which converge to markedly impair mitochondrial energy utilization and ATP production.

### SUMMARY

This document relates to methods and materials for treating hypoxic conditions (e.g., tissue ischemia). For example, this document relates to compositions containing a potato polysaccharide preparation and organic selenium to increase or maintain mitochondrial NO formation. In some cases, compositions provided herein also can contain inorganic nitrate.

Producing intra-mitochondrial NO can result in a protective effect against hypoxic conditions such as tissue ischemia. Having the ability to use a composition containing a potato polysaccharide preparation and organic selenium provided herein to increase or maintain mitochondrial NO formation can provide clinicians and patients with an effective treatment regime for treating hypoxic conditions (e.g., tissue ischemia) and/or diseases associated with hypoxia.

This document also provides compositions (e.g., nutritional supplement compositions) that contain a potato polysaccharide preparation and organic selenium, and, optionally, inorganic nitrate. For example, this document provides nutritional supplement compositions containing a potato polysaccharide preparation and organic selenium, and, optionally, inorganic nitrate; methods for making nutritional supplement compositions containing a potato polysaccharide preparation and organic selenium, and, optionally, inorganic nitrate; and methods for increasing or decreasing expression of polypeptides involved in mitochondrial NO formation.

A composition containing a potato polysaccharide preparation and an organic selenium can include between about 1 mg and about 250 mg of potato polysaccharide preparation (e.g., between about 5 mg and about 30 mg of said potato polysaccharide preparation). For example, a composition containing a potato polysaccharide preparation and an organic selenium can include about 10 mg of said potato polysaccharide preparation. The organic selenium can be L-selenomethionine. A composition containing a potato polysaccharide preparation and an organic selenium can include between about 10 μg and about 650 μg L-selenomethionine (e.g., between about 160 μg and about 180 μg L-selenomethionine). For example, a composition containing a potato polysaccharide preparation and an organic selenium can include about 175 μg L-selenomethionine.

A composition containing a potato polysaccharide preparation and an organic selenium can also include an inorganic nitrate. A composition containing a potato polysaccharide preparation and an organic selenium can include between about 10 mg and about 750 mg nitrate (e.g., between about 250 mg and about 400 mg nitrate). For example, a composition containing a potato polysaccharide preparation and an organic selenium can include about 350 mg nitrate.

A composition containing a potato polysaccharide preparation and an organic selenium can be in the form of a tablet.

A composition containing a potato polysaccharide preparation and an organic selenium can be a nutritional supplement.

A composition containing a potato polysaccharide preparation and an organic selenium can be formulated to deliver to a mammal about 0.15 mg potato polysaccharide preparation, about 2.9 μg of organic selenium, and about 5.7 mg of inorganic nitrate, per kg of body weight of the mammal.

In some aspects, this document includes methods for treating a hypoxic condition. Such methods can include administering a composition containing a potato polysaccharide preparation and an organic selenium to a mammal having a hypoxic condition, such that a symptom of hypoxic condition is reduced. The hypoxic condition can be tissue ischemia. A composition administered to a mammal having a hypoxic condition can also include an inorganic nitrate. For example, a composition administered to a mammal having a hypoxic condition can include a potato polysaccharide preparation in an amount that results in between about 0.10 mg and about 0.2 mg (e.g., about 0.15 mg) of the potato polysaccharide preparation being administered to said mammal per kg of body weight of said mammal, organic selenium in an amount that results in between about 2.5 μg and about 3.5 μg (e.g., about 2.9 μg) of the organic selenium being administered to said mammal per kg of body weight of said mammal, and inorganic nitrate in an amount that results in between about 3 mg and about 9 mg (e.g., about 5.7 mg) of the inorganic nitrate being administered to said mammal per kg of body weight of said mammal. The composition can be in the form of a tablet. The mammal can be a human.

In some aspects, this document includes methods for treating a disease associated with a hypoxic condition. Such methods can include administering a composition containing a potato polysaccharide preparation and an organic selenium to a mammal having a disease associated with a hypoxic condition, such that a symptom of the disease associated with the hypoxic condition is reduced. The disease associated with the hypoxic condition can be tissue ischemia, type 2 diabetes, cardiovascular disease, chronic obstructive pulmonary disease, end stage renal disease, fatty liver disease, or Alzheimer's disease. A composition administered to a mammal having a disease associated with a hypoxic condition can also include an inorganic nitrate. For example, a composition administered to a mammal having a disease associated with a hypoxic condition can include a potato polysaccharide preparation in an amount that results in between about 0.10 mg and about 0.2 mg (e.g., about 0.15 mg) of the potato polysaccharide preparation being administered to said mammal per kg of body weight of said mammal, organic selenium in an amount that results in between about 2.5 μg and about 3.5 μg (e.g., about 2.9 μg) of the organic selenium being administered to said mammal per kg of body weight of said mammal, and inorganic nitrate in an amount that results in between about 3 mg and about 9 mg (e.g., about 5.7 mg) of the inorganic nitrate being administered to said mammal per kg of body weight of said mammal. The composition can be in the form of a tablet. The mammal can be a human.

In some aspects, this document includes methods for increasing mitochondrial NO formation in cells. Such methods include contacting cells with a composition comprising a potato polysaccharide preparation and an organic selenium, such that mitochondrial NO formation the cells is increased. The composition can also an inorganic nitrate. The cells can be neuronal, vascular, cardiac, kidney, cutaneous, muscle, adipocytes, or digestive cells. The cells can be human cells.

In some aspects, this document includes methods increasing polypeptide expression in cells. Such methods can include contacting cells with a composition containing a potato polysaccharide preparation, organic selenium, and inorganic nitrate, such that expression of one or more polypeptides involved in mitochondrial NO formation is increased. The composition can also an inorganic nitrate. For example, cells can be contacted with a composition containing a potato polysaccharide preparation in an amount that results in between about 0.10 mg and about 0.2 mg (e.g., about 0.15 mg) of the potato polysaccharide preparation being administered to said mammal per kg of body weight of said mammal, organic selenium in an amount that results in between about 2.5 μg and about 3.5 μg (e.g., about 2.9 μg) of the organic selenium being administered to said mammal per kg of body weight of said mammal, and inorganic nitrate in an amount that results in between about 3 mg and about 9 mg (e.g., about 5.7 mg) of the inorganic nitrate being administered to said mammal per kg of body weight of said mammal. The cells can be neuronal, vascular, cardiac, kidney, cutaneous, muscle, adipocytes, or digestive cells. The cells can be human cells. The polypeptide(s) can be one or more nitrate reductases. The polypeptide(s) can be XDH, NFE2L2, ABCB6, SLC48A1, COA3, COA4, COX17, COX6C, COX7A2, COX7B, ATP7B, CCS, ATOX1, MOCS2, COX10, COX19, or COX8A.

In some aspects, this document provides a nutritional supplement containing a potato polysaccharide preparation, L-selenomethionine, and an inorganic nitrate. The nutritional supplement can be in the form of a tablet. The nutritional supplement can include potato polysaccharide preparation in an amount that results in between about 0.10 mg and about 0.2 mg (e.g., about 0.15 mg) of the potato polysaccharide preparation being administered to said mammal per kg of body weight of said mammal, organic selenium in an amount that results in between about 2.5 μg and about 3.5 μg (e.g., about 2.9 μg) of the organic selenium being administered to said mammal per kg of body weight of said mammal, and inorganic nitrate in an amount that results in between about 3 mg and about 9 mg (e.g., about 5.7 mg) of the inorganic nitrate being administered to said mammal per kg of body weight of said mammal. The nutritional supplement can contain about 10 mg of said potato polysaccharide preparation. The nutritional supplement can contain about 175 μg L-selenomethionine. The nutritional supplement can contain about 350 mg inorganic nitrate.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic outline of an administration schedule. Abbreviations: body weight (BW); liquid nitrogen (LN2); milligrams per kilogram (mg/kg); per os, oral gavage (PO); once a day (QD); whole blood (WB).

### DETAILED DESCRIPTION

This document provides methods and materials related to using compositions containing a potato polysaccharide preparation and organic selenium to treat hypoxic conditions (e.g., tissue ischemia). For example, this document provides compositions containing a potato polysaccharide preparation and organic selenium, and, optionally, inorganic nitrate; as well as methods of using such compositions.

A composition provided herein (e.g., a nutritional supplement) can contain a potato polysaccharide preparation and organic selenium, and, optionally, inorganic nitrate. As described herein, a composition containing a potato polysaccharide preparation and organic selenium provided herein (e.g., a nutritional supplement) can be administered to any appropriate mammal to treat the mammal for a hypoxic condition, to treat the mammal for a disease or disorder associated with a hypoxic condition, to increase mitochondrial NO formation, and/or to increase expression of polypeptides involved in mitochondrial NO formation. Examples of hypoxic conditions that can be treated as described herein include, without limitation, tissue ischemia (e.g., acute tissue ischemia), tissue hypo-perfusion, vascular insufficiency, mitochondrial impairment, metabolic rundown, and oxidative stress. Examples of diseases or disorders associated with a hypoxic condition include, without limitation, type 2 diabetes, cardiovascular disease, chronic obstructive pulmonary disease (COPD), end stage renal disease, fatty liver disease, Alzheimer's disease (AD), atherosclerosis, rheumatoid arthritis, peripheral artery disease (PAD), ischemic bowel disease, mesenteric ischemia, and angina pectoris.

A composition provided herein can include any appropriate potato polysaccharide. For example, a compositions provided herein can include a potato polysaccharide preparation described in WO 2013/148282. In some cases, a potato polysaccharide preparation included in a composition provided herein can be a chemically characterized xyloglucan purified from extracted raw potato (i.e., a “potosaccharide”). A potato polysaccharide preparation can be a preparation that is obtained from a water extract of potato and that contains polysaccharide material having the ability to be eluted from a C18 cartridge (e.g., a Sep-Pak Plus C-18 cartridge) with 10% acetonitrile. In some cases, a potato polysaccharide preparation can be a preparation that is obtained from potato and that contains polysaccharide material having HPLC characteristics of that of the peak eluted at 3.5 minutes. In some cases, a polysaccharide of a potato polysaccharide preparation provided herein can be a polar, water-soluble polysaccharide. In some cases, a polysaccharide of a potato polysaccharide preparation provided herein can be a highly substituted complex xyloglucan material.

In some cases, a composition provided herein can be designed to include a potato polysaccharide preparation that is obtained from potato and that contains polysaccharide material that, when derivatized and assessed using GC/MS, results in at least four major components (3,4-furan dimethanol, diacetate; 1,2,3,4,5-penta-o-acetyl-D-xylitol (isomer 1); 3,5-diacetoxybenzyl alcohol; and D-glucitol-hexaacetate).

In some cases, a potato polysaccharide preparation included in a composition provided herein can be a preparation that is obtained from potato and that contains polysaccharide material that, when derivatized, results in at least the following acylated carbohydrates as assessed using GC/MS: (a) myo-inositol (set to 1× to serve as an internal standard), (b) glucose at about 40× to about 60× the myo-inositol content (e.g., glucose at about 50× the myo-inositol content), (c) xylose at about 10× to about 20× the myo-inositol content (e.g., xylose at about 15× the myo-inositol content), (d) mannose at about 5× to about 15× the myo-inositol content (e.g., mannose at about 10× the myo-inositol content), and (e) galactose at about 3× to about 7× the myo-inositol content (e.g., galactose at about 5× the myo-inositol content). The derivatization procedure can include forming a dry residue of the polysaccharide material that is then hydrolyzed using trifluoroacetic acid. The resulting material is then reduced using sodium borohydride, and after borate removal, the end product is acylated using acetic anhydride and pyridine. The end products of the reaction are then injected directly on GC/MS to identify the acylated carbohydrates.

In some cases, a potato polysaccharide preparation included in a composition provided herein can be a substantially pure potato polysaccharide preparation. Typically, a substantially pure potato polysaccharide preparation is a preparation that contains a single peak of material (e.g., a single peak of polysaccharide material) when assessed using, for example, HPLC. In some cases, greater than 60, 70, 75, 80, 85, 90, 95, or 99 percent of a potato polysaccharide preparation provided herein can be polysaccharide material obtained from a potato.

Any appropriate potato species or variety can be used to obtain a potato polysaccharide preparation included in a composition provided herein. For example, Solanum tuberosum, Ipomoea batatas, S. acaule, S. bukasovii, S. leptophyes, S. megistacrolobum, S. commersonii, or S. infundibuliforme can be used to obtain a potato polysaccharide preparation provided herein. In some cases, potato varieties of S. tunerosum such as Organic Yellow, Purple or blue varieties, Cream of the Crop, Adirondack Blue, Adirondack Red, Agata, Almond, Andes Gold, Andes Sun, Apline, Alturas, Amandine, Annabelle, Anya, Arran Victory, Atlantic, Avalanche, Bamberg, Bannock Russet, Belle de Fontenay, BF-15, Bildtstar, Bintje, Blazer Russet, Blue Congo, Bonnotte, British Queens, Cabritas, Camota, Canela Russet, Cara, Carola, Chelina, Chiloé, Cielo, Clavela Blanca, Desiree, Estima, Fianna, Fingerling, Flava, German Butterball, Golden Wonder, Goldrush, Home Guard, Innovator, Irish Cobbler, Jersey Royal, Kennebec, Kerr's Pink, Kestrel, Keuka Gold, King Edward, Kipfler, Lady Balfour, Langlade, Linda, Marcy, Marfona, Maris Piper, Marquis, Megachip, Monalisa, Nicola, Pachacoña, Pike, Pink Eye, Pink Fir Apple, Primura, Ranger Russet, Ratte, Record, Red LaSoda, Red Norland, Red Pontiac, Rooster, Russet Burbank, Russet Norkotah, Selma, Shepody, Sieglinde, Silverton Russet, Sirco, Snowden, Spunta, Up to date, Stobrawa, Superior, Vivaldi, Vitelotte, Yellow Finn, or Yukon Gold can be used to obtain a potato polysaccharide preparation included in a composition provided herein.

Any appropriate dose of a potato polysaccharide preparation provided herein can be used to formulate a composition containing a potato polysaccharide preparation and organic selenium provided herein (e.g., a nutritional supplement). For example, a composition containing a potato polysaccharide preparation and organic selenium provided herein can contain between about 1 mg and about 750 mg (e.g., between about 1.5 mg and about 600 mg, between about 2 mg and about 500 mg, between about 2.5 mg and about 400 mg, between about 3 mg and about 300 mg, between about 3.5 mg and about 200 mg, between about 4 mg and about 100 mg, between about 4.5 mg and about 50 mg, or between about 5 mg and about 20 mg) of the potato polysaccharide component of the composition. For example, a composition containing a potato polysaccharide preparation and organic selenium provided herein can contain between about 7 mg and about 10 mg potato polysaccharide preparation. In some cases, a composition containing a potato polysaccharide and organic selenium (e.g., a nutritional supplement) can be formulated to deliver about 0.15 mg of the potato polysaccharide component per kg of body weight to a mammal (e.g., a human) per day. For example, a nutritional supplement can be formulated into a single oral composition that a human can swallow once a day to provide about 0.15 mg of the potato polysaccharide preparation per kg of body weight.

A composition provided herein can be designed to include any appropriate organic selenium. For example, the organic selenium can be present in a compound. Examples of organic selenium compounds that can be included within a composition provided herein include, without limitation, dimethyl selenide, selenomethionine, selenocysteine, and methylselenocysteine. The organic selenium can be present in any enantiomeric form. For example, a selenomethionine can be L-selenomethionine.

Any appropriate dose of organic selenium provided herein can be used to formulate a composition containing a potato polysaccharide preparation and organic selenium provided herein (e.g., a nutritional supplement). Organic selenium compounds are toxic in large doses. The organic selenium can be a low, non-toxic concentration of L-selenomethionine. For example, a composition containing a potato polysaccharide preparation and organic selenium provided herein can contain between about 0.05 nM and about 1 nM (e.g., between about 0.06 nM and about 0.9 nM, between about 0.07 nM and about 0.8 nM, between about 0.08 nM and about 0.7 nM, between about 0.09 nM and about 0.6 nM, between about 0.1 nM and about 0.5 nM, or between about 0.2 nM and about 0.4 nM) of the organic selenium component of the composition. For example, a composition containing a potato polysaccharide preparation and organic selenium provided herein can contain between about 1 μg and about 750 μg (e.g., between about 10 μg and about 650 μg, between about 25 μg and about 600 μg, between about 50 μg and about 500 μg, between about 75 μg and about 400 μg, between about 100 μg and about 300 μg, between about 125 μg and about 250 μg, or between about 150 μg and about 200 μg) of the organic selenium component of the composition. For example, a composition containing a potato polysaccharide preparation and organic selenium provided herein can contain between about 160 μg and about 180 μg selenium. In some cases, a composition containing a potato polysaccharide preparation and organic selenium (e.g., a nutritional supplement) provided herein can be formulated to deliver about 2.9 μg of organic selenium per kg of body weight to a mammal (e.g., a human) per day. For example, a nutritional supplement can be formulated into a single oral composition that a human can swallow once a day to provide about 2.9 μg of organic selenium per kg of body weight.

A composition containing a potato polysaccharide preparation and organic selenium can contain other ingredients such as inorganic nitrate, calcium, and/or arginine (e.g., L-arginine). For example, a composition containing a potato polysaccharide preparation and organic selenium also can contain an inorganic nitrate. An inorganic nitrate can include any appropriate inorganic nitrate. The inorganic nitrate can be present in a compound (e.g., a salt). Examples of inorganic nitrates that can be included in a composition provided herein include, without limitation, sodium nitrate and potassium nitrate. The inorganic nitrate can be a low, non-toxic concentration of inorganic nitrate. In some embodiments, the inorganic nitrate is a low, non-toxic concentration of sodium nitrate. Any appropriate dose of inorganic nitrate provided herein can be used to formulate a composition containing a potato polysaccharide preparation and organic selenium provided herein (e.g., a nutritional supplement). For example, a composition containing a potato polysaccharide preparation, organic selenium, and inorganic nitrate provided herein can contain between about 10 mg and about 750 mg (e.g., between about 15 mg and about 700 mg, between about 25 mg and about 650 mg, between about 50 mg and about 600 mg, between about 75 mg and about 550 mg, between about 100 mg and about 500 mg, between about 150 mg and about 450 mg, between about 200 mg and about 425 mg, or between about 250 mg and about 400 mg) of the inorganic nitrate component of the composition. For example, a composition containing a potato polysaccharide preparation, organic selenium, and inorganic nitrate provided herein can contain between about 300 mg and about 375 mg of inorganic nitrate. In some cases, a composition (e.g., a nutritional supplement) containing a potato polysaccharide, organic selenium, and inorganic nitrate can be formulated to deliver about 5.7 mg of the inorganic nitrate component per kg of body weight to a mammal (e.g., a human) per day. For example, a nutritional supplement can be formulated into a single oral composition that a human can swallow once a day to provide about 5.7 mg of inorganic nitrate per kg of body weight. For example, a composition containing a potato polysaccharide preparation and organic selenium also can contain arginine (e.g., L-arginine). L-arginine can be included to promote a cell's ability to release nitric oxide via nitric oxide synthesis from L-arginine metabolism. The composition comprises between about 1 mg and about 500 mg (e.g., between about 25 mg and about 475 mg, between about 50 mg and about 450 mg, between about 100 mg and about 425 mg, between about 150 mg and about 400 mg, or between about 200 mg and about 350 mg) of arginine. In some cases, a composition containing a potato polysaccharide preparation and organic selenium also can contain about 300 mg L-arginine. For example, a composition containing a potato polysaccharide preparation and organic selenium also can contain calcium. Calcium sources such as calcium citrate or CaCO₃ can be added to help facilitate the metabolism of L-arginine into nitric oxide via a calcium-dependent constitutive nitric oxide synthase. To reduce acid reflux problems in oral applications, CaCO₃ can be used as a calcium source. The composition can contain between about 250 μg and about 1.5 g (e.g., between about 300 μg and about 1.45 g, between about 500 μs and about 1.4 g, between about 750 μg and about 1.35 g, or between 1 g and 1.3 g) of the calcium source. In some cases, a composition containing a potato polysaccharide preparation and organic selenium also can contain between 1 g and 1.3 g calcium.

In some cases, a composition containing a potato polysaccharide preparation and organic selenium can be formulated together with one or more additional ingredients (e.g., inorganic nitrate, calcium, and/or arginine) to form a single composition. In some cases, additional ingredients (e.g., inorganic nitrate, calcium, and/or arginine) can be provided to a mammal in a separate composition; one containing a potato polysaccharide preparation and organic selenium, and one containing, for example, inorganic nitrate.

Any method can be used to obtain a potato polysaccharide preparation, organic selenium, and/or or any additional component (e.g., inorganic nitrate) of compositions provided herein. In some cases, the components of the compositions provided herein can be obtained using common chemical extraction, isolation, or synthesis techniques. In some cases, a potato polysaccharide preparation can be obtained as described in WO 2013/148282. For example, a potato polysaccharide preparation can be obtained by homogenizing raw potato material in water and maintaining the homogenate at room temperature for a period of time (e.g., about 1 hour) with occasional shaking. The homogenate can be centrifuged (e.g., at 4000 g for 10 minutes) to remove any larger solid material. The resulting supernatant can be loaded onto a Solid Phase Extraction cartridge (e.g., a C18 cartridge such as a Sep-Pak Plus C-18 cartridge), and the polysaccharide material eluted with 10 percent acetonitrile. Once eluted, the polysaccharide material can be dried and stored (e.g., at about 4° C.). In some cases, the components of the compositions provided herein can be obtained from commercial vendors. For example, organic selenium and inorganic nitrate can be ordered from Sigma, Inc.

In some cases, a composition containing a potato polysaccharide preparation and organic selenium, and, optionally, inorganic nitrate can be formulated as a pharmaceutical composition and/or a nutritional supplement (e.g., a “nutraceutical”). For example, a composition containing a potato polysaccharide and organic selenium, and, optionally, inorganic nitrate provided herein can contain a pharmaceutically and/or nutritionally acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers for oral administration.

An acceptable aqueous vehicle can be, for example, any liquid solution that is capable of dissolving a composition containing a potato polysaccharide preparation and organic selenium, and, optionally, inorganic nitrate provided herein and is not toxic to the particular individual receiving the composition. Examples of acceptable aqueous vehicles include, without limitation, saline, water, and acetic acid. Typically, acceptable aqueous vehicles are sterile. An acceptable solid vehicle can be formulated such that compositions containing a potato polysaccharide preparation and organic selenium, and, optionally, inorganic nitrate provided herein is suitable for oral administration. The dose supplied by each capsule or tablet can vary since an effective amount can be reached by administrating either one or multiple capsules or tablets. Any appropriate pharmaceutically or nutritionally acceptable material such as gelatin and cellulose derivatives can be used as an acceptable solid vehicle. In addition, an acceptable solid vehicle can be a solid carrier including, without limitation, starch, sugar, or bentonite. Further, a tablet or pill formulation of a composition containing a potato polysaccharide preparation and organic selenium, and, optionally, inorganic nitrate can follow conventional procedures that employ solid carriers, lubricants, and the like. In some cases, a formulation of a composition containing a potato polysaccharide preparation and organic selenium, and, optionally, inorganic nitrate can be formulated for controlled release.

Any appropriate method can be used to formulate a pharmaceutical composition and/or nutritional supplement provided herein (e.g., a pharmaceutical composition containing a potato polysaccharide preparation and organic selenium, and, optionally, inorganic nitrate provided herein). For example, common formulation mixing and preparation techniques can be used to make a composition having the components described herein. In addition, the compositions provided herein can be in any appropriate form. For example, a composition provided herein can be in the form of a solid, liquid, and/or aerosol including, without limitation, powders, crystalline substances, gels, pastes, ointments, salves, creams, solutions, suspensions, partial liquids, sprays, nebulae, mists, atomized vapors, tinctures, pills, capsules, tablets, and gelcaps. In some cases, the composition can be a dietary supplement. In some embodiments, compositions containing a potato polysaccharide preparation and organic selenium, and, optionally, inorganic nitrate provided herein can be prepared for oral administration by mixing the components with one or more of the following: a filler, a binder, a disintegrator, a lubricant, and a coloring agent. Lactose, corn starch, sucrose, glucose, sorbitol, crystalline cellulose, silicon dioxide, or the like can be used as the filler. Polyvinyl alcohol, polyvinyl ether, ethyl cellulose, methyl cellulose, acacia, tragacanth, gelatin, shellac, hydroxypropyl cellulose, hydroxypropylmethyl cellulose, calcium citrate, dextrin, or pectin can be used as the binder. Magnesium stearate, talc, polyethylene glycol, silica, or hardened plant oil can be used as the lubricant. A pharmaceutically acceptable coloring agent can be used as the coloring agent. Cocoa powder, mentha water, aromatic acid, mentha oil, borneol, or powdered cinnamon bark also can be added. In some cases, compositions containing a potato polysaccharide preparation and organic selenium, and, optionally, inorganic nitrate provided herein can be prepared for injection by mixing the components with one or more of the following: a pH adjusting agent, a buffer, a stabilizer, and a solubilizing agent.

This document also provides methods and materials for using a composition (e.g., a nutritional supplement) containing a potato polysaccharide preparation and organic selenium, and, optionally, inorganic nitrate provided herein. For example, compositions provided herein can be used to treat a mammal having a hypoxic condition (e.g., tissue ischemia), to treat a mammal having a disease or disorder associated with a hypoxic condition, to increase or maintain mitochondrial NO formation in cells, and/or to increase polypeptide expression (e.g., polypeptides involved in mitochondrial NO formation) in cells.

Methods for treating a mammal having a hypoxic condition (e.g., tissue ischemia) and/or methods for treating a mammal having a disease or disorder associated with a hypoxic condition can include administering to the mammal a composition (e.g., a nutritional supplement) including a potato polysaccharide preparation and organic selenium, and, optionally, inorganic nitrate. Examples of diseases and disorders associated with hypoxic conditions that can be treated using the compositions containing a potato polysaccharide preparation and organic selenium, and optionally, inorganic nitrate provided herein include, without limitation, type 2 diabetes, cardiovascular disease, COPD, end stage renal disease, fatty liver disease, AD, atherosclerosis, rheumatoid arthritis, PAD, ischemic bowel disease, mesenteric ischemia, and angina pectoris. Methods for treating a mammal having a hypoxic condition and/or methods for treating a mammal having a disease or disorder associated with a hypoxic condition can be effective to reduce or eliminate one or more symptoms of a hypoxic condition. Examples of tissue ischemia symptoms include, without limitation, pain, pallor, pulseless, paresthesia, paralysis, poikilothermia, cyanosis, cardiac arrhythmia, and atrial fibrillation. Methods for treating a mammal having a hypoxic condition can include identifying the mammal as having a hypoxic condition. Methods for treating a mammal having a disease or disorder associated with a hypoxic condition can include identifying the mammal as having a disease or disorder associated with a hypoxic condition. Once identified as having a hypoxic condition or having a disease or disorder associated with a hypoxic condition, the mammal can be administered or instructed to self-administer a composition (e.g., a nutritional supplement) having a potato polysaccharide preparation and organic selenium, and, optionally, inorganic nitrate.

In some cases, methods for treating a mammal having a hypoxic condition and/or methods for treating a mammal having a disease or disorder associated with a hypoxic conditions can include additional treatment such as injection of an anticoagulant (e.g., heparin), thrombolysis (e.g., catheter-directed thrombolysis), embolectomy, arteriotomy, surgical revascularisation, amputation, and/or decreasing body temperature.

Compositions containing a potato polysaccharide preparation and organic selenium, and, optionally, inorganic nitrate provided herein can be administered to any mammal (e.g., rat, mouse, dog, cat, horse, cow, goat, pig, monkey, or human). In addition, any route of administration (e.g., oral or parenteral administration) can be used to administer a composition provided herein to a mammal. For example, compositions provided herein can be administered orally or parenterally (e.g., a subcutaneous, intramuscular, intraorbital, intracapsular, intraspinal, intrasternal, or intravenous injection).

Any appropriate dose of a composition (e.g., a nutritional supplement) containing a potato polysaccharide preparation, organic selenium, and, optionally, inorganic nitrate provided herein can be used to formulate pharmaceuticals provided herein (e.g., a pharmaceutical including a composition containing a potato polysaccharide preparation and organic selenium, and, optionally, inorganic nitrate). For example, a composition provided herein (e.g., a nutritional supplement) can be formulated to contain about 7 mg to about 10 mg of potato polysaccharide preparation, about 160 μg to about 180 μg of selenium (e.g., organic selenium such as L-selenomethionine), and optionally, about 300 mg to about 375 mg of nitrate (e.g., inorganic nitrate). For example, a composition provided herein (e.g., a nutritional supplement) can be formulated to deliver about 0.15 mg of potato polysaccharide preparation, about 2.9 μg of organic selenium, and optionally, about 5.7 mg of inorganic nitrate, per kg of body weight to a mammal (e.g., a human) per day. Various factors can influence the actual amount used for a particular application. For example, the frequency of administration, duration of treatment, combination of other agents, site of administration, stage of disease (if present), and the anatomical configuration of the treated area may require an increase or decrease in the actual amount administered.

The frequency of administration of compositions (e.g., nutritional supplements) containing a potato polysaccharide preparation and organic selenium, and, optionally, inorganic nitrate provided herein can be any frequency. For example, the frequency of administration can be from about four times a day to about once a month, or more specifically, from about twice a day to about once a week. For example, a composition provided herein (e.g., a nutritional supplement) can be formulated into a single oral composition that a mammal (e.g., a human) can swallow once a day to provide about 0.15 mg of potato polysaccharide preparation, about 2.9 μg of organic selenium, and optionally, about 5.7 mg of inorganic nitrate, per kg of body weight. For example, a composition provided herein (e.g., a nutritional supplement) can be formulated into a single oral composition that a human can swallow once a day to provide about 7 mg to about 10 mg of potato polysaccharide preparation, about 160 μg to about 180 μg of selenium (e.g., organic selenium such as L-selenomethionine), and optionally, about 300 mg to about 375 mg of nitrate (e.g., inorganic nitrate). In addition, the frequency of administration can remain constant or can be variable during the duration of treatment. As with the amount administered, various factors can influence the actual frequency of administration used for a particular application. For example, the amount (dose), duration of treatment, combination of agents, site of administration, stage of disease (if present), and the anatomical configuration of the treated area may require an increase or decrease in administration frequency.

The duration of administration of a composition (e.g., a nutritional supplement) containing a potato polysaccharide preparation and organic selenium, and, optionally, inorganic nitrate provided herein can be any duration. For example, a duration of administration of compositions provided herein can be longer than a week, month, three months, six months, nine months, a year, two years, or three years. In some cases, an effective duration can be any duration that reduces, prevents, or eliminates a symptom of a disease upon administration to a mammal without producing significant toxicity to the mammal. Such an effective duration can vary from several days to several weeks, months, or years. In general, an effective duration for the treatment of an acute disease can range in duration from several days to several months. Once administration of compositions containing a potato polysaccharide preparation and organic selenium, and, optionally, inorganic nitrate provided herein is stopped, however, disease symptoms may return. In such cases, an effective duration for the prevention of certain conditions can last for as long as the individual is alive. Multiple factors can influence the actual duration used for a particular treatment or prevention regimen. For example, an effective duration can vary with the frequency of administration, the amount administered, combination of multiple agents, site of administration, state of disease (if present), and anatomical configuration of the treated area.

Methods for increasing or maintaining mitochondrial NO formation in cells and/or for increasing polypeptide expression (e.g., polypeptides involved in mitochondrial NO formation) in cells can include contacting the cells with a composition containing a potato polysaccharide preparation and organic selenium, and, optionally, inorganic nitrate. Cells can be in vitro or in vivo. Cells can be from any appropriate sources (e.g., mammalian cells such as human cells). In addition, the cells can be any type of cell including, without limitation, neuronal, vascular, cardiac, kidney, cutaneous, muscle, adipocytes, and/or digestive cells. A composition containing a potato polysaccharide preparation and organic selenium, and, optionally, inorganic nitrate provided herein can be contacted with the cells by any appropriate method. A composition containing a potato polysaccharide preparation and organic selenium, and, optionally, inorganic nitrate provided herein can be contacted with cells in an amount and at a frequency such that mitochondrial NO formation the cells is increased. A composition containing a potato polysaccharide preparation and organic selenium, and, optionally, inorganic nitrate provided herein can be contacted with cells in an amount and at a frequency such that expression of one or more polypeptides (e.g., polypeptides involved in mitochondrial NO formation) is increased. For example, a composition containing a potato polysaccharide preparation and organic selenium, and, optionally, inorganic nitrate provided herein can be used to increase expression of one of more polypeptides (e.g., polypeptides involved in mitochondrial NO formation) by at least 1.2 fold (e.g., at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, or at least 2.5 fold). For example, a composition containing a potato polysaccharide preparation and organic selenium, and, optionally, inorganic nitrate provided herein can be used to increase expression of one of more polypeptides (e.g., polypeptides involved in mitochondrial NO formation) by at least 20 percent (e.g., at least 30 percent, at least 40 percent, at least 50 percent, at least 60 percent, at least 70 percent, at least 80 percent, at least 90 percent, at least 100 percent, at least 110 percent, at least 120 percent, at least 130 percent, at least 140 percent, at least 150 percent, at least 160 percent, at least 170 percent, at least 180 percent, at least 190 percent, or at least 200 percent).

While not being limited to any particular mode of action, compositions (e.g., nutritional supplements) containing a potato polysaccharide preparation and organic selenium, and, optionally, inorganic nitrate provided herein can be used to increase or maintain mitochondrial NO formation via polypeptides involved in mitochondrial NO formation (e.g., nitrite reductases such as molybdenum-dependent and COX heme/copper-dependent nitrite reductases). A composition containing a potato polysaccharide preparation and organic selenium, and, optionally, inorganic nitrate provided herein can be used to increase expression of one or more polypeptides involved in mitochondrial NO formation (e.g., nitrite reductases such as molybdenum-dependent and COX heme/copper-dependent nitrite reductases). For example, a composition provided herein can be used to increase expression of xanthine dehydrogenase/xanthine oxidoreductase (XDH); nuclear factor, erythroid 2-like 2 (NFE2L2); ATP-binding cassette, sub-family B (MDR/TAP), member 6 (Langereis blood group) (ABCB6); solute carrier family 48 (heme transporter), member 1 (SLC48A1); cytochrome C oxidase assembly factor 3 (COA3); cytochrome c oxidase assembly factor 4 (COA4); cytochrome c oxidase assembly homolog 17 (COX17); cytochrome c oxidase, subunit Vic (COX6C); cytochrome c oxidase subunit VIIa polypeptide 2 (COX7A2); cytochrome c oxidase subunit VIIb (COX7B); ATPase, Cu++ transporting, beta polypeptide (ATP7B); copper chaperone for superoxide dismutase (CCS); ATX1 antioxidant protein 1 homolog (ATOX1); molybdenum cofactor synthesis 2 (MOCS2); COX10 homolog, cytochrome c oxidase assembly protein, heme A: farnesyltransferase (COX10); COX19 cytochrome c oxidase assembly homolog (COX19); cytochrome c oxidase subunit VIIIA (COX8A); or a combination thereof. In some cases, a composition containing a potato polysaccharide preparation and organic selenium, and, optionally, inorganic nitrate provided herein can be used to increase expression of one or more polypeptides involved in mitochondrial NO formation (e.g., one or more of XDH, NFE2L2, ABCB6, SLC48A1, COA3, COA4, COX17, COX6C, COX7A2, COX7B, ATP7B, CCS, ATOX1, MOCS2, COX10, COX19, and COX8A).

In some cases, a composition containing a potato polysaccharide preparation and organic selenium, and, optionally, inorganic nitrate provided herein can be used to increase expression of a XDH polypeptide by about 1.2 fold to about 5 fold (e.g., by about 1.3 to about 2.5 fold, by about 1.4 to about 2 fold, by about 1.6 fold). In some cases, a composition containing a potato polysaccharide preparation and organic selenium, and, optionally, inorganic nitrate provided herein can be used to increase expression of a NFE2L2 polypeptide by about 1.2 fold to about 5 fold (e.g., by about 1.2 to about 2 fold, by about 1.2 to about 1.5 fold, by about 1.3 fold). In some cases, a composition containing a potato polysaccharide preparation and organic selenium, and, optionally, inorganic nitrate provided herein can be used to increase expression of a ABCB6 polypeptide by about 1.2 fold to about 5 fold (e.g., by about 1.5 to about 4 fold, by about 2 to about 3 fold, by about 2.2 fold). In some cases, a composition containing a potato polysaccharide preparation and organic selenium, and, optionally, inorganic nitrate provided herein can be used to increase expression of a SLC48A1 polypeptide by about 1.2 fold to about 5 fold (e.g., by about 1.5 to about 4 fold, by about 1.7 to about 3 fold, by about 2 fold). In some cases, a composition containing a potato polysaccharide preparation and organic selenium, and, optionally, inorganic nitrate provided herein can be used to increase expression of a COA3 polypeptide by about 1.2 fold to about 5 fold (e.g., by about 1.3 to about 3.5 fold, by about 1.4 to about 2 fold, by about 1.5 fold). In some cases, a composition containing a potato polysaccharide preparation and organic selenium, and, optionally, inorganic nitrate provided herein can be used to increase expression of a COA4 polypeptide by about 1.2 fold to about 5 fold (e.g., by about 1.2 to about 2.5 fold, by about 1.2 to about 2 fold, by about 1.3 fold). In some cases, a composition containing a potato polysaccharide preparation and organic selenium, and, optionally, inorganic nitrate provided herein can be used to increase expression of a COX17 polypeptide by about 1.2 fold to about 5 fold (e.g., by about 1.5 to about 4 fold, by about 2 to about 3 fold, by about 2.3 fold). In some cases, a composition containing a potato polysaccharide preparation and organic selenium, and, optionally, inorganic nitrate provided herein can be used to increase expression of a COX6C polypeptide by about 1.2 fold to about 5 fold (e.g., by about 1.3 to about 4 fold, by about 1.5 to about 3 fold, by about 1.8 fold). In some cases, a composition containing a potato polysaccharide preparation and organic selenium, and, optionally, inorganic nitrate provided herein can be used to increase expression of a COX7A2 polypeptide by about 1.2 fold to about 5 fold (e.g., by about 1.5 to about 4 fold, by about 2 to about 3 fold, by about 2.2 to about 2.4 fold). In some cases, a composition containing a potato polysaccharide preparation and organic selenium, and, optionally, inorganic nitrate provided herein can be used to increase expression of a COX7B polypeptide by about 1.2 fold to about 5 fold (e.g., by about 1.3 to about 4 fold, by about 1.5 to about 3 fold, by about 1.8 fold). In some cases, a composition containing a potato polysaccharide preparation and organic selenium, and, optionally, inorganic nitrate provided herein can be used to increase expression of a ATP7B polypeptide by about 1.2 fold to about 5 fold (e.g., by about 1.3 to about 2.5 fold, by about 1.5 to about 2 fold, by about 1.7 fold). In some cases, a composition containing a potato polysaccharide preparation and organic selenium, and, optionally, inorganic nitrate provided herein can be used to increase expression of a CCS polypeptide by about 1.2 fold to about 5 fold (e.g., by about 1.5 to about 4.5 fold, by about 2 to about 3 fold, by about 2.4 fold). In some cases, a composition containing a potato polysaccharide preparation and organic selenium, and, optionally, inorganic nitrate provided herein can be used to increase expression of a ATOX1 polypeptide by about 1.2 fold to about 5 fold (e.g., by about 1.2 to about 4 fold, by about 1.3 to about 3 fold, by about 1.4 to about 2.5 fold). In some cases, a composition containing a potato polysaccharide preparation and organic selenium, and, optionally, inorganic nitrate provided herein can be used to increase expression of a MOCS2 polypeptide by about 1.2 fold to about 5 fold (e.g., by about 1.5 to about 4 fold, by about 2 to about 3 fold, by about 2.2 fold). In some cases, a composition containing a potato polysaccharide preparation and organic selenium, and, optionally, inorganic nitrate provided herein can be used to increase expression of a COX10 polypeptide by about 1.2 fold to about 5 fold (e.g., by about 1.2 to about 2.5 fold, by about 1.3 to about 2 fold, by about 1.4 fold). In some cases, a composition containing a potato polysaccharide preparation and organic selenium, and, optionally, inorganic nitrate provided herein can be used to increase expression of a COX19 polypeptide by about 1.2 fold to about 5 fold (e.g., by about 1.2 to about 3 fold, by about 1.2 to about 2 fold, by about 1.3 fold). In some cases, a composition containing a potato polysaccharide preparation and organic selenium, and, optionally, inorganic nitrate provided herein can be used to increase expression of a COX8A polypeptide by about 1.2 fold to about 5 fold (e.g., by about 1.3 to about 3 fold, by about 1.4 to about 2 fold, by about 1.5 fold).

In humans, a composition containing a potato polysaccharide preparation and organic selenium, and optionally, inorganic nitrate, provided herein can be used to decrease expression of a human XDH polypeptide, a human NFE2L2 polypeptide, a human ABCB6 polypeptide, a human SLC48A1 polypeptide, a human COA3 polypeptide, a human COA4 polypeptide, a human COX17 polypeptide, a human COX6C polypeptide, a human COX7A2 polypeptide, a human COX7B polypeptide, a human ATP7B polypeptide, a human CCS polypeptide, a human ATOX1 polypeptide, a human MOCS2 polypeptide, a human COX10 polypeptide, a human COX19 polypeptide, a human COX8A polypeptide, or a combination thereof.

In some cases, a human XDH polypeptide can have an amino acid sequence set forth in, for example, National Center for Biotechnology Information (NCBI) Accession Nos: AAA75287.1 (see, e.g., GI No. 984267), BAA02013.2 (see, e.g., GI No. 10336525), EAX00479.1 (see, e.g., GI No. 119620884), and AAY68219.1 (see, e.g., GI No. 67515423). In some cases, a human NFE2L2 polypeptide can have an amino acid sequence set forth in, for example, NCBI Accession Nos: NP_001300833.1 (see, e.g., GI No. 926657654), NP_001300832.1 (see, e.g., GI No. 926657652), NP_001300831.1 (see, e.g., GI No. 926657650), NP_001300830.1 (see, e.g., GI No. 926657648), NP_001300829.1 (see, e.g., GI No. 926657646), NP_001138885.1 (See, e.g., GI No. 224028259), NP_001138884.1 (see, e.g., GI No. 224028257), and NP_006155.2 (see, e.g., GI No. 20149576). In some cases, a human ABCB6 polypeptide can have an amino acid sequence set forth in, for example, NCBI Accession Nos: AAH43423.1 (see, e.g., GI No. 71297293), NP_005680.1 (see, e.g., GI No. 9955963), EAW70705.1 (see, e.g., GI No. 119591111), EAW70704.1 (see, e.g., GI No. 119591110), EAW70703.1 (see, e.g., GI No. 119591109), EAW70702.1 (see, e.g., GI No. 119591108), EAW70701.1 (see, e.g., GI No. 119591107), EAW70700.1 (see, e.g., GI No. 119591106), EAW70699.1 (see, e.g., GI No. 119591105), EAW70698.1 (see, e.g., GI No. 119591104), EAW70696.1 (see, e.g., GI No. 119591102), EAW70695.1 (see, e.g., GI No. 119591101), EAW70694.1 (see, e.g., GI No. 119591100), AAH00559.1 (see, e.g., GI No. 12653571), and BAN91500.2 (see, e.g., GI No. 591985814). In some cases, a human SLC48A1 polypeptide can have an amino acid sequence set forth in, for example, NCBI Accession Nos: AAH65033.1 (see, e.g., GI No. 40675612), AAH02759.2 (see, e.g., GI No. 31581976), and AAH26344.2 (see, e.g., GI No. 133777216). In some cases, a human COA3 polypeptide can have an amino acid sequence set forth in, for example, NCBI Accession No: NP_001035521.1 (see, e.g., GI No. 94536771). In some cases, a human COA4 polypeptide can have an amino acid sequence set forth in, for example, NCBI Accession No: NP_057649.2 (see, e.g., GI No. 46198304). In some cases, a human COX17 polypeptide can have an amino acid sequence set forth in, for example, NCBI Accession Nos: AAA98114.1 (see, e.g., GI No. 1280206), AAF82569.1 (see, e.g., GI No. 9049966), AAI08318.1 (see, e.g., GI No. 80478662), AAI05281.1 (see, e.g., GI No. 75867819), AAH10933.1 (see, e.g., GI No. 15012067), and NP_005685.1 (see, e.g., GI No. 5031645). In some cases, a human COX6C polypeptide can have an amino acid sequence set forth in, for example, NCBI Accession Nos: NP_004365.1 (see, e.g., GI No. 4758040), EAW91793.1 (see, e.g., GI No. 119612199), EAW91792.1 (see, e.g., GI No. 119612198), EAW91791.1 (see, e.g., GI No. 119612197), AAC73061.1 (see, e.g., GI No. 3859868), and AAH00187.1 (see, e.g., GI No. 12652867). In some cases, a human COX7A2 polypeptide can have an amino acid sequence set forth in, for example, NCBI Accession Nos: CAG46922.1 (see, e.g., GI No. 49457246), CAG28574.1 (see, e.g., GI No. 47115229), AAI33655.1 (see, e.g., GI No. 126632021), NP_001856.2 (see, e.g., GI No. 262118227), AAI00855.1 (see, e.g., GI No. 72533460), AAI00853.1 (see, e.g., GI No. 71682267), AAI00854.1 (see, e.g., GI No. 71680896), EAW48747.1 (see, e.g., GI No. 119569132), EAW48746.1 (see, e.g., GI No. 119569131), AAI01827.1 (see, e.g., GI No. 75517299), AAI01829.1 (see, e.g., GI No. 75516916), and CCF23109.1 (see, e.g., GI No. 377549892). In some cases, a human COX7B polypeptide can have an amino acid sequence set forth in, for example, NCBI Accession Nos: CAG46921.1 (see, e.g., GI No. 49457244), CAG29328.1 (see, e.g., GI No. 47496611), NP_001857.1 (see, e.g., GI No. 4502991), EAW98607.1 (see, e.g., GI No. 119619013), and AAH18386.1 (see, e.g., GI No. 17390909). In some cases, a human ATP7B polypeptide can have an amino acid sequence set forth in, for example, NCBI Accession Nos: AAB52902.1 (see, e.g., GI No. 1947035), NP_001230111.1 (see, e.g., GI No. 342187274), NP_001005918.1 (see, e.g., GI No. 55743073), NP_000044.2 (see, e.g., GI No. 55743071), EAX08896.1 (see, e.g., GI No. 119629301), EAX08895.1 (see, e.g., GI No. 119629300), EAX08894.1 (see, e.g., GI No. 119629299), EAX08893.1 (see, e.g., GI No. 119629298), and EAX08892.1 (see, e.g., GI No. 119629297). In some cases, a human CCS polypeptide can have an amino acid sequence set forth in, for example, NCBI Accession Nos: NP_005116.1 (see, e.g., GI No. 4826665), AAC51764.1 (see, e.g., GI No. 2431868), AAI12056.1 (see, e.g., GI No. 85567355), AAI05017.1 (see, e.g., GI No. 85397493), AAM50090.1 (see, e.g., GI No. 21429608), EAW74550.1 (see, e.g., GI No. 119594956), EAW74551.1 (see, e.g., GI No. 119594957), and EAW74549.1 (see, e.g., GI No. 11959495). In some cases, a human ATOX1 polypeptide can have an amino acid sequence set forth in, for example, NCBI Accession Nos: CAG33182.1 (see, e.g., GI No. 48145919), NP_004036.1 (see, e.g., GI No. 4757804), EAW61664.1 (see, e.g., GI No. 119582068), EAW61663.1 (see, e.g., GI No. 119582067), and AAX81411.1 (see, e.g., GI No. 62361685). In some cases, a human MOCS2 polypeptide can have an amino acid sequence set forth in, for example, NCBI Accession Nos: CAG33453.1 (see, e.g., GI No. 48146461), NP_004522.1 (see, e.g., GI No. 4758732), EAW54877.1 (see, e.g., GI No. 119575272), EAW54876.1 (see, e.g., GI No. 119575271), EAW54875.1 (see, e.g., GI No. 119575270), EAW54874.1 (see, e.g., GI No. 119575269), AAH46097.1 (see, e.g., GI No. 28278189), and AAD14599.1 (see, e.g., GI No. 4262373). In some cases, a human COX10 polypeptide can have an amino acid sequence set forth in, for example, NCBI Accession Nos: EAW89957.1 (see, e.g., GI No. 119610363), EAW89956.1 (see, e.g., GI No. 119610362), AAH06394.1 (see, e.g., GI No. 13623563), AAH00060.1 (see, e.g., GI No. 12652629), and AAP35631.1 (see, e.g., GI No. 30582809). In some cases, a human COX19 polypeptide can have an amino acid sequence set forth in, for example, NCBI Accession Nos: AAY35062.1 (see, e.g., GI No. 63253780), NP_001026788.1 (see, e.g., GI No. 71979925), AAH70383.1 (see, e.g., GI No. 127799539), AAI10421.1 (see, e.g., GI No. 114205607), and AAI03633.1 (see, e.g., GI No. 73909196). In some cases, a human COX8A polypeptide can have an amino acid sequence set forth in, for example, NCBI Accession Nos: EAW74186.1 (see, e.g., GI No. 119594592), NP_004065.1 (see, e.g., GI No. 4758044), and AAH63025.1 (see, e.g., GI No. 38614453).

The invention will be further described in the following examples, which do not limit the scope of the invention described in the claims.

### EXAMPLES

### Example 1: In Vivo Administration of Potato Polysaccharide and Selenium

A formulation for enhancing mitochondrial NO formation from reserves of inorganic nitrite via the concerted actions of molybdenum-dependent and COX heme/copper-dependent nitrite reductases was designed. In vivo administration of a low concentration of a highly pure and chemically characterized polysaccharide extracted from raw potato, here termed potosaccharide, to genetically obese Zucker ZDF rats engendered concerted changes in the expression of major blood leukocyte genes functionally linked to clinically significant regulation of mitochondrial oxygen utilization via the actions of molybdenum-dependent and COX heme/copper-dependent nitrite reductases.

### Materials and Methods

Extraction and purification of potosaccharide. Typically, 6 g of potato were homogenized with a Polytron homogenizer in 20 mL water in a 50 mL centrifuge tube and kept at room temperature for 1 hour. The homogenate was centrifuged at 4000 rpm for 10 minutes and the supernatant fraction was reserved. 10 mL of the supernatant fraction was percolated through a Sep-Pak Plus C-18 cartridge previously activated with 10 mL 100% acetonitrile (ACN) followed by 10 mL 0.05% trifluoroacetic acid in water (TFA water). Following successive low ACN washes, semi-purified potosaccharide was eluted in 10 mL 10% ACN in 0.05% TFA water. The eluent fraction was dried and reconstituted in 1 mL 0.05% TFA water for further purification via HPLC.

The reconstituted 10% ACN eluent fraction was subjected to HPLC purification utilizing a Waters Xterra RP C18 column (4.6×150 mm) and Waters 2695 separations module with a photodiode array detector. HPLC purification employed a shallow 20 minute gradient ranging from 0 to 2.5% in 0.05% TFA water at a flow rate of 0.5 mL/min. Collection and HPLC re-purification of a major 198 nm UV absorbing peak at 3.5 minute yielded a symmetrical HPLC peak containing highly purified potosaccharide. The purified HPLC fraction was dried and reconstituted in phosphate buffered saline (PBS) for use in biological experiments.

Experimental Animals.

Twenty-two 7-week old, male Zucker Diabetic Fatty rats (ZDF, Code: 370) and twenty-two 7-8 week old, male ZDF Lean rats (Code: 371) were purchased from Charles Rivers Laboratories (Wilmington, Mass.). The study animals were allowed an acclimation period of 4 days prior to baseline blood collections, at which time two extra animals from each strain were dropped from the study based on baseline body weight. The rats were housed 2 per cage and maintained in the Innovive caging system (San Diego, Calif.) upon arrival at PhysioGenix, Inc. Cages were monitored daily to ensure the Innovive system maintained 80 air changes per hour and positive pressure. In accordance with the Guide for Care and Use of Laboratory Animals (Eighth Edition), rat rooms were maintained at temperatures of 66-75 degrees Fahrenheit and relative humidity between 30% and 70%. The rooms were lit by artificial light for 12 hours each day (7:00 AM-7:00 PM). Animals had free access to water and Purina 5008 rodent food (Waldschimdt's, Madison, Wis.) for the duration of the study except during fasted experiments.

Potosaccharide Formulation.

Purified potosaccharide (10 mL stock solution at 5 mg/mL concentration) was stored at 4° C. The vehicle for the study was sterile water (Catalog number 002488, Butler Schein). Each week, the stock solution was diluted 1:100 in sterile water (0.05 mg/mL) and dispensed into daily aliquots. All vehicle and drug solutions were stored at 4° C. and administered at room temperature daily by oral gavage (PO) in a volume of 1 mL/animal (0.15 mg/kg dose based on estimated body weight of 350 grams).

Dosing and Grouping.

Two types of rats were used for the study: homozygous obese ZDF/ZDF and heterozygous lean littermates. The rats within the groups were then chosen at random and divided into groups of 10. Group 1 was the homozygous ZDF/ZDF vehicle fed rats, group 2 was the homozygous ZDF/ZDF potosaccharide fed, group 3 was the lean vehicle fed rat and group 4 was the lean potosaccharide fed rats. The vehicle was distilled water and the potosaccharide was given daily each morning via oral gavage at a dosage of 0.05 mg per animal. The dose was usually given in 1 mL of water. Rats were caged in groups and maintained in 12 hours light/12 hours dark (7 am-7 pm). The study lasted for 28 days and all animals were euthanized by isoflurane overdose and thoracotomy following the collection of fasted blood glucose data on Day 28 of the Study. Blood was collected via descending vena cava. Liver and abdominal fat were collected, weighed, and a portion of the left lateral liver lobe and abdominal fat were placed into individual histology cassettes and snap frozen in liquid nitrogen. General pathological observations were recorded.

Primary Cell Cultures.

Embryonic chicken kidney and liver cells were obtained from Charles River Laboratories, Inc. Cells were cultured in 75 cc flasks. Each flask represented a treatment group as follows: Control, 1 nM selenomethionine. Treatments consisted of 24 hour incubations with selenomethionine. After that period, samples were processed for microarray analysis. Experiments were performed in triplicate.

RNA Isolation.

Total RNA extracted from rat tissue samples was isolated and purified using the RNeasy mini kit (Qiagen, Valencia, Calif.). Typically, 100 mg of tissue was resuspended in 1.8 mL of RLT lysis buffer (Qiagen) and homogenized with a polytron homogenizer for 30 seconds. Blood RNA was isolated using the PAX RNA kit (Qiagen).

DNA Microarray Analyses.

DNA microarray analyses were performed using a system provided by Agilent. Arrays included four arrays per chip (Agilent 4×44K chip). Total RNA was reverse transcribed (1000 ng) using T7 primers and labeled and transcribed using Cyanine-3 dye. Each array was hybridized with at least 1.65 μg of labeled cRNA at 65° C. for 18 hours. Arrays were scanned using an Agilent array scanner. The microarray platform can determine a minimum of a 1.5 fold change in gene expression. Fold changes between homozygous ZDF rats fed potosaccharide for 28 days vs homozygous ZDF rats fed an equivalent amount of vehicle (control) were examined. In addition, gene expression was measured in heterozygous ZDF (lean rats) fed with vehicle only and compared to gene expression in homozygous ZDF control rats. Fold changes were used to calculate a percent restoration by using the fold changes between homozygous ZDF rats fed potosaccharide and lean control rats.

### Results

As depicted in Table 1, in vivo administration of purified potosaccharide to ZDF rats (n=7) vs vehicle control ZDF rats engendered statistically significant enhanced expression of four high profile candidate genes that are involved in synergistic enhancement of mitochondrial NO formation from reserves of inorganic nitrite via the concerted actions of molybdenum-dependent and COX heme/copper-dependent nitrite reductases. The four candidate genes include XDH (Xanthine dehydrogenase/xanthine oxidoreductase), NFE2L2 (nuclear factor, erythroid 2-like 2 ABCB6 ATP-binding cassette, sub-family B, member 6), and SLC48A1 (solute carrier family 48 [heme transporter], member 1). In dramatic fashion, in vivo potosaccharide treatment restored the aberrant gene expression of the homozygous ZDF rats to near normal lean control values with a high degree of statistical significance (86-100% restoration of normative gene function).

TABLE 1
Enhanced expression of nuclear-encoded mitochondrial nitrite reductase genes previously
associated with major pro-inflammatory and hypoxic disease states, as monitored in
blood samples of ZDF rats vs vehicle control ZDF rats (n = 7) following in vivo
potosaccharide administration. Data sets were derived by DNA microarray analyses.

| | | Fold Change | | % |
|---|---|---|---|---|
| Gene Symbol | Gene Name | (Poto) | p value | restored |
| XDH | Xanthine dehydrogenase/xanthine oxidoreductase | 1.6 | 0.04 | 86 |
| NFE2L2 | nuclear factor, erythroid 2-like 2 | 1.3 | 0.01 | 100 |
| ABCB6 | ATP-binding cassette, sub-family B (MDR/TAP), member 6 | 2.2 | 0.001 | 100 |
| | (Langereis blood group) | | | |
| SLC48A1 | solute carrier family 48 (heme transporter), member 1 | 2.0 | 0.003 | 94 |

As depicted in Table 2 below, in vivo administration of purified potosaccharide to ZDF rats (n=7) vs vehicle control ZDF rats engendered statistically significant enhanced expression of nine high profile candidate genes that are involved in synergistic enhancement of mitochondrial NO formation from reserves of inorganic nitrite via the enhanced actions of COX heme/copper-dependent nitrite reductases. The nine candidate genes include COA3 and COA4 (cytochrome C oxidase assembly factors 3 and 4), COX17 (cytochrome c oxidase assembly homolog 17), COX6C (cytochrome c oxidase, subunit VIc), COX7A2 (cytochrome c oxidase subunit VIIa polypeptide 2), COX7B (cytochrome c oxidase subunit VIIb), ATP7B (ATPase, Cu++ transporting, beta polypeptide), CCS (copper chaperone for superoxide dismutase), and ATOX1 (ATX1 antioxidant protein 1 homolog). In dramatic fashion, in vivo potosaccharide treatment restored the aberrant gene expression of the homozygous ZDF rats to near normal lean control values with a high degree of statistical significance (53-100% restoration of normative gene function).

TABLE 2
Enhanced expression of nuclear-encoded COX/copper-related mitochondrial genes previously
associated with major pro-inflammatory and hypoxic disease states, as monitored in
blood samples of ZDF rats vs vehicle control ZDF rats (n = 7) following in vivo
potosaccharide administration. Data sets were derived by DNA microarray analyses.

| | | Fold Change | | % |
|---|---|---|---|---|
| Gene Symbol | Gene Name | (Poto) | p value | restored |
| COA3 | cytochrome C oxidase assembly factor 3 | 1.5 | 0.004 | 87 |
| COA4 | cytochrome c oxidase assembly factor 4 | 1.3 | 0.01 | 62 |
| COX17 | cytochrome c oxidase assembly homolog 17 (yeast) | 2.3 | 0.0003 | 78 |
| COX6C | cytochrome c oxidase, subunit VIc | 1.8 | 0.0004 | 100 |
| COX7A2 | cytochrome c oxidase subunit VIIa polypeptide 2 | 2.4 | 0.0008 | 100 |
| COX7B | cytochrome c oxidase subunit VIIb | 1.8 | 0.005 | 100 |
| ATP7B | ATPase, Cu++ transporting, beta polypeptide | 1.7 | 0.008 | 100 |
| CCS | copper chaperone for superoxide dismutase | 2.4 | 0.0002 | 53 |
| ATOX1 | ATX1 antioxidant protein 1 homolog | 2.2 | 0.0002 | 56 |

As depicted in Table 3 below, in vitro incubation of embryonic chicken liver cells with a low, non-toxic, 1 nM concentration of L-selenomethionine resulted in enhanced expression of MOCS2 and five nuclear-encoded COX/copper-related mitochondrial genes previously associated with major pro-inflammatory and hypoxic disease states. The five nuclear-encoded candidate genes include ATOX1, COX10 (COX10 homolog, cytochrome c oxidase assembly protein, heme A: farnesyltransferase), COX19 (COX19 cytochrome c oxidase assembly homolog, COX7A2 (cytochrome c oxidase subunit VIIa polypeptide 2), COX8A (cytochrome c oxidase subunit VIIIa).

TABLE 3
Enhanced expression of nuclear-encoded COX/copper-related mitochondrial genes previously associated with
major pro-inflammatory and hypoxic disease states, as monitored in chicken liver cells incubated with
1 nM selenomethione vs vehicle treated cells. Data sets were derived by DNA microarray analyses.

| | | | Fold Change |
|---|---|---|---|
| Probe Name | Gene Symbol | Gene Name | (1 nM Se vs Control) |
| A_87_P034931 | MOCS2 | molybdenum cofactor synthesis 2 | 2.2 |
| A_87_P034931 | ATOX1 | ATX1 antioxidant protein 1 homolog | 1.4 |
| A_87_P057796 | COX10 | COX10 homolog, cytochrome c oxidase assembly protein, | 1.4 |
| | | heme A: farnesyltransferase (yeast) | |
| A_87_P071146 | COX19 | COX19 cytochrome c oxidase assembly homolog | 1.3 |
| | | (S. cerevisiae) | |
| A_87_P115183 | COX7A2 | cytochrome c oxidase subunit VIIa polypeptide 2 (liver) | 2.2 |
| A_87_P088916 | COX8A | cytochrome c oxidase subunit VIIIA (ubiquitous) | 1.5 |

These results demonstrate that both a potosaccharide and low, non-toxic concentrations of L-selenmethionine enhanced medically beneficial mitochondrial NO formation. These results also demonstrate potosaccharide-mediated enhancement of gene expression of COX heme/copper-dependent nitrite reductases that can facilitate mitochondrial NO production. Taken together, these results demonstrate that a composition containing a potato polysaccharide preparation and an organic selenomethionine can be used to treat acute tissue ischemia.

### Example 2: In Vivo Administration of Potato Saccharide, Selenium, and Nitrate

As described herein, a composition of potosaccharide in combination with low, non-toxic concentrations of L-selenomethionine enhanced mitochondrial NO formation from reserves of inorganic nitrite. The addition of inorganic nitrate to enhance cellular nitrite reserves can be used to provide additional production of mitochondrial NO.

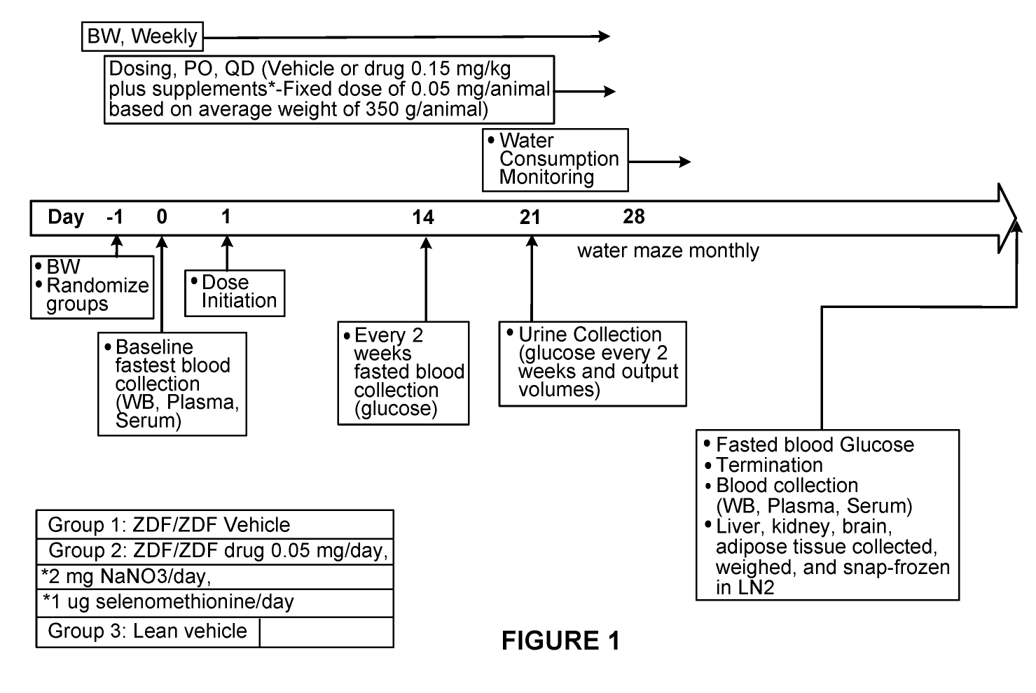

Animals are administered a potato polysaccharide preparation, selenium, and/or nitrate as shown in FIG. 1.

### Materials and Methods

Preparation Formulation.

Purified preparation (10 mL stock solution at 5 mg/mL concentration) is stored at 4° C. The vehicle for the study is sterile water. Each week, the stock solution is diluted 1:100 in sterile water (0.05 mg/mL) and dispensed into daily aliquots. The formulation is modified to contain 1 μg selenomethionine and/or 2 mg sodium nitrate per mL. All vehicle and drug solutions are stored at 4° C. and administered at room temperature daily by oral gavage (PO) in a volume of 1 mL/animal (0.15 mg/kg dose based on estimated body weight of 350 grams).

Experimental Animals.

Two types of rats are used for the study: homozygous obese ZDF/ZDF and heterozygous lean littermates. Thirty, 7-week old, male Zucker Diabetic Fatty rats (ZDF, Code: 370) are purchased from Charles Rivers Laboratories (Wilmington, Mass.). Fifteen heterozygous lean rats are obtained from the same source. The study animals are allowed an acclimation period of 4 days prior to baseline blood collections. The rats are housed two per cage, maintained in the Innovive caging system (San Diego, Calif.) and are monitored daily to ensure the Innovive system maintained 80 air changes per hour and positive pressure. In accordance with the Guide for Care and Use of Laboratory Animals (Eighth Edition), rat rooms are maintained at temperatures of 66-75° F. and relative humidity between 30% and 70%. The rooms are lit by artificial light for 12 hours each day (7:00 AM-7:00 PM). Animals have free access to water and Purina 5008 rodent food for the duration of the study except during fasted experiments.

Dosing and Grouping.

The homozygous obese ZDF/ZDF rats are chosen at random and divided into 2 groups of 15. Group 1 is the homozygous ZDF/ZDF vehicle fed rats, group 2 is the homozygous ZDF/ZDF purified preparation fed. Group 3 is 15 heterozygous lean, vehicle fed rats. The vehicle is distilled water and the supplemented purified preparation is given daily each morning via oral gavage at a dosage of 0.05 mg purified preparation, 1 μg selenomethionine and 2 mg sodium nitrate per animal. The dose is given in 1 mL of water. Rats are caged in groups and maintained in 12 hours light/12 hours dark (7 am-7 pm). The study is for a minimum of 12 weeks with termination determined by ongoing data collection and analysis. At termination, all animals are euthanized by isoflurane overdose and thoracotomy following the collection of fasted blood glucose data.

Specimen and Data Collection.

Blood is collected via descending vena cava every 2 weeks starting 2 weeks post-dosing and subjected to glucose analysis and serum creatinine analysis. Urine is collected every 2 weeks starting 3 weeks post-dosing and subjected to proteinuria analysis and urine creatinine analysis. Total creatinine and creatinine clearance are also determined. Liver, kidney, brain, and abdominal fat are collected at termination, weighed, and tissue samples are placed into individual histology cassettes and snap frozen in liquid nitrogen. General pathological observations are recorded.

Assays.

Whole genome microarrays are performed with the frozen tissue samples. Whole blood is preserved in PAX RNA blood tubes for gene expression analysis. Cognition of the study animals is assessed monthly using the Morris water maze or similar methodology. Alternatively the Biel water maze or the radial arm water maze may be used. Biel water maze records time to completion and number of errors. Morris records time and path length.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A composition comprising a potato polysaccharide preparation and an organic selenium.

2. The composition of claim 1, wherein said composition comprises between about 1 mg and about 250 mg of potato polysaccharide preparation.

3-4. (canceled)

5. The composition of claim 1, wherein said organic selenium is L-selenomethionine.

6. The composition of claim 5, wherein said composition comprises between about 10 μg and about 650 μg L-selenomethionine.

7-8. (canceled)

9. The composition of claim 1, wherein said composition comprises an inorganic nitrate.

10. The composition of claim 9, wherein said composition comprises between about 10 mg and about 750 mg nitrate.

11-12. (canceled)

13. The composition of claim 1, wherein said composition is in the form of a tablet.

14. The composition of claim 1, wherein said composition is a nutritional supplement.

15. The composition of claim 9, wherein said composition is formulated to deliver to a mammal about 0.15 mg potato polysaccharide preparation, about 2.9 μg of organic selenium, and about 5.7 mg of inorganic nitrate, per kg of body weight of the mammal.

16. A method for treating a hypoxic condition, the method comprising:
administering to a mammal having said hypoxic condition a composition comprising a potato polysaccharide preparation and an organic selenium;
wherein a symptom of said hypoxic condition is reduced.

17. The method of claim 16, wherein said hypoxic condition comprises tissue ischemia.

18. The method of claim 16, wherein said composition comprises an inorganic nitrate.

19. The method of claim 18, wherein said composition comprises said potato polysaccharide preparation in an amount that results in between about 0.10 mg and about 0.2 mg of the potato polysaccharide preparation being administered to said mammal per kg of body weight of said mammal, wherein said composition comprises said organic selenium in an amount that results in between about 2.5 μg and about 3.5 μg of the organic selenium being administered to said mammal per kg of body weight of said mammal, and wherein said composition comprises said inorganic nitrate in an amount that results in between about 3 mg and about 9 mg of the inorganic nitrate being administered to said mammal per kg of body weight of said mammal.

20. (canceled)

21. The method of claim 16, wherein said composition is in the form of a tablet.

22. The method of claim 16, wherein said mammal is a human.

23. A method for treating a disease associated with a hypoxic condition, the method comprising:
administering to a mammal having said disease associated with said hypoxic condition a composition comprising a potato polysaccharide preparation and an organic selenium;
wherein a symptom of said disease associated with said hypoxic condition is reduced.

24. The method of claim 23, wherein said disease associated with the hypoxic condition is selected from the group consisting of tissue ischemia, type 2 diabetes, cardiovascular disease, chronic obstructive pulmonary disease (COPD), end stage renal disease, fatty liver disease, and Alzheimer's disease (AD).

25. The method of claim 23, wherein said composition comprises an inorganic nitrate.

26. The method of claim 25, wherein said composition comprises said potato polysaccharide preparation in an amount that results in between about 0.10 mg and about 0.2 mg of the potato polysaccharide preparation being administered to said mammal per kg of body weight of said mammal, wherein said composition comprises said organic selenium in an amount that results in between about 2.5 μg and about 3.5 μg of the organic selenium being administered to said mammal per kg of body weight of said mammal, and wherein said composition comprises said inorganic nitrate in an amount that results in between about 3 mg and about 9 mg of the inorganic nitrate being administered to said mammal per kg of body weight of said mammal.

27. (canceled)

28. The method of claim 23, wherein said composition is in the form of a tablet.

29. The method of claim 23, wherein said mammal is a human.

30-48. (canceled)
